(19) United States
(12) Patent Application Publication
(10) Pub. No.: US 2022/0395193 A1
(43) Pub. Date: Dec. 15, 2022

(54) HEIGHT ESTIMATION APPARATUS, HEIGHT ESTIMATION METHOD, AND NON-TRANSITORY COMPUTER READABLE MEDIUM STORING PROGRAM

(71) Applicant: NEC Corporation, Minato-ku, Tokyo (JP)

(72) Inventors: Noboru YOSHIDA, Tokyo (JP); Shoji NISHIMURA, Tokyo (JP)

(73) Assignee: NEC Corporation, Minato-ku, Tokyo (JP)

(21) Appl. No.: 17/619,011

(22) PCT Filed: Jun. 26, 2019
(86) PCT No.: PCT/JP2019/025269

§ 371 Date: Dec. 14, 2021

(51) Int. Cl. A61B 5/107; G06V 40/10; G06T 7/60; G06T 7/70

(52) U.S. Cl. CPC A61B 5/1072; G06V 40/103; G06T 7/60; G06T 7/70; G06T 2207/20044; G06T 2207/30196; G06V 2201/07

(57) ABSTRACT

A height estimation apparatus (10) according to the present disclosure includes an acquisition unit (11) for acquiring a two-dimensional image obtained by capturing an animal, a detection unit (12) for detecting a two-dimensional skeletal structure of the animal based on the two-dimensional image acquired by the acquisition unit (11), and an estimation unit (13) for estimating a height of the animal in a three-dimensional real world based on the two-dimensional skeletal structure detected by the detection unit (12) and an imaging parameter of the two-dimensional image acquired by the acquisition unit (11).

### TECHNICAL FIELD

The present disclosure relates to a height estimation apparatus, a height estimation method, and a non-transitory computer readable medium storing a program.

### BACKGROUND ART

Recently, a technique in which an image of an animal such as a person is captured by a camera and an attribute of the person or the like is recognized from the captured image has been used. As a technique related to estimation of a height which is an attribute of a person or the like, for example, Patent Literature 1 to 3 is known. Patent Literature 1 describes a technique for estimating a height of a person based on a length of a long side or lengths of the long side and a short side of a person area in an image. Patent Literature 2 describes a technique for estimating a height of a person based on a distance image. Patent Literature 3 describes a technique for estimating a height using an imaging result captured by an X-ray CT apparatus. In addition, Non Patent Literature 1 is known as a technique related to skeleton estimation of a person.

### CITATION LIST

### Patent Literature

Patent Literature 1: International Patent Publication No. WO 2017/209089
Patent Literature 2: Japanese Unexamined Patent Application Publication No. 2012-120647
Patent Literature 3: Japanese Unexamined Patent Application Publication No. 2012-231816

### Non Patent Literature

Non Patent Literature 1: Zhe Cao, Tomas Simon, Shih-En Wei, Yaser Sheikh, “Realtime Multi-Person 2D Pose Estimation using Part Affinity Fields”, The IEEE Conference on Computer Vision and Pattern Recognition (CVPR), 2017, P. 7291-7299

### SUMMARY OF INVENTION

### Technical Problem

As described above, in Patent Literature 1, since the height is estimated based on a size of the person area in the image, estimation accuracy of the height may be lowered depending on a posture of the person and an orientation of the person with respect to the camera. Further, in Patent Literature 2, it is essential to acquire the distance image, and in Patent Literature 3, a special contrast imaging has to be performed by an X-ray CT apparatus. For these reasons, there is a problem in the related art that it is difficult to accurately estimate the height from a two-dimensional image obtained by capturing the animal such as a person.

In view of such a problem, it is an object of the present disclosure to provide a height estimation apparatus, a height estimation method, and a non-transitory computer readable medium storing a program capable of improving accuracy of estimating a height.

### Solution to Problem

A height estimation apparatus according to the present disclosure includes: acquisition means for acquiring a two-dimensional image obtained by capturing an animal; detection means for detecting a two-dimensional skeletal structure of the animal based on the acquired two-dimensional image; and estimation means for estimating a height of the animal in a three-dimensional real world based on the detected two-dimensional skeletal structure and an imaging parameter of the two-dimensional image.

A height estimation method according to the present disclosure includes: acquiring a two-dimensional image obtained by capturing an animal; detecting a two-dimensional skeletal structure of the animal based on the acquired two-dimensional image; and estimating a height of the animal in a three-dimensional real world based on the detected two-dimensional skeletal structure and an imaging parameter of the two-dimensional image.

A non-transitory computer readable medium storing a program according to the present disclosure for causing a computer to execute processing of: acquiring a two-dimensional image obtained by capturing an animal; detecting a two-dimensional skeletal structure of the animal based on the acquired two-dimensional image; and estimating a height of the animal in a three-dimensional real world based on the detected two-dimensional skeletal structure and an imaging parameter of the two-dimensional image.

### Advantageous Effects of Invention

According to the present disclosure, it is possible to provide a height estimation apparatus, a height estimation method, and a non-transitory computer readable medium storing a program capable of improving accuracy of estimating a height.

### BRIEF DESCRIPTION OF DRAWINGS

FIG. 1 is a flowchart showing a monitoring method according to related art;

FIG. 2 is a block diagram showing an overview of a height estimation apparatus according to example embodiments;

FIG. 3 is a block diagram showing a configuration of a height estimation apparatus according to a first example embodiment;

FIG. 4 is a flowchart showing a height estimation method according to the first example embodiment;

FIG. 5 is a flowchart showing a height pixel count calculation method according to the first example embodiment;

FIG. 6 shows a human body model according to the first example embodiment;

FIG. 7 shows an example of detection of a skeletal structure according to the first example embodiment;

FIG. 8 shows an example of detection of the skeletal structure according to the first example embodiment;

FIG. 9 shows an example of detection of the skeletal structure according to the first example embodiment;

FIG. 10 shows a human body model according to a second example embodiment;

FIG. 11 is a flowchart showing a height pixel count calculation method according to the second example embodiment;

FIG. 12 shows an example of detection of the skeletal structure according to the second example embodiment;

FIG. 13 is a histogram for explaining a height pixel count calculation method according to the second example embodiment;

FIG. 14 is a flowchart showing a height estimation method according to a third example embodiment;

FIG. 15 shows an example of detection of a skeletal structure according to the third example embodiment;

FIG. 16 shows a three-dimensional human body model according to the third example embodiment;

FIG. 17 is a diagram for explaining the height estimation method according to the third example embodiment;

FIG. 18 is a diagram for explaining the height estimation method according to the third example embodiment;

FIG. 19 is a diagram for explaining the height estimation method according to the third example embodiment; and

FIG. 20 is a block diagram showing an overview of hardware of a computer according to the example embodiments.

### DESCRIPTION OF EMBODIMENTS

Example embodiments will be described below with reference to the drawings. In each drawing, the same elements are denoted by the same reference signs, and the repeated description is omitted if necessary.

### (Study Leading to Example Embodiments)

Recently, image recognition technology utilizing machine learning has been applied to various systems. As an example, a monitoring system for performing monitoring using images captured by a monitoring camera will be discussed.

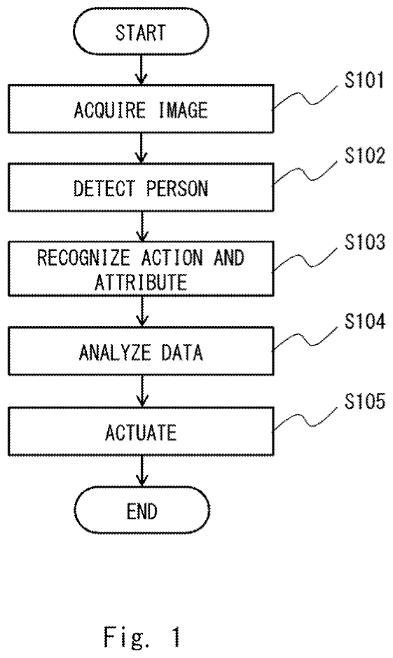

FIG. 1 shows a monitoring method performed by a monitoring system according to related art. As shown in FIG. 1, the monitoring system acquires an image from the monitoring camera (S101), detects a person from the acquired image (S102), and performs action recognition and attribute recognition of the person (S103). For example, a behavior and a movement line of the person are recognized as the actions of the person, and age, gender, height, etc. of the person are recognized as the attributes of the person. Further, the monitoring system performs data analysis on the recognized actions and attributes of the person (S104), and actuation such as processing based on an analysis result or the like is performed (S105). For example, the monitoring system displays an alert from the recognized actions, and the attribute such as the recognized height of the person is monitored.

As shown in this example, there is a growing demand for easily obtaining attribute information such as age, gender, and height of a person from images or videos of a monitoring camera. Among these attributes, the height is useful information for identifying individuals and distinguishing adults from children. For example, the attribute information is used for investigation as characteristics of a criminal, such as 30s, male, 170 cm, for marketing as information of customers, and for searching for a lost child as a characteristic of the lost child.

As a result of a study on a method for recognizing a height of a person from an image by the inventors, they found that the related technique cannot always recognize or estimate the height accurately. For example, when a whole body of a person appears in the image, the height can be estimated to some extent. However, the person in the image is not always upright, or the top of the head and the foot do not always appear in the image. Especially in the case of a lost children, there is a high possibility that he/she is crouching down. In such cases, it is difficult to estimate the height.

Therefore, the inventors studied a method using a skeleton estimation technique by means of machine learning for estimating a height of a person. For example, in a skeleton estimation technique according to related art such as OpenPose disclosed in Non Patent Literature 1, a skeleton of a person is estimated by learning various patterns of annotated image data. In the following example embodiments, a height of a person can be accurately estimated by utilizing such a skeleton estimation technique.

The skeletal structure estimated by the skeleton estimation technique such as OpenPose is composed of “key points” which are characteristic points such as joints, and “bones, i.e., bone links” indicating links between the key points. Therefore, in the following example embodiments, the skeletal structure is described using the terms “key point” and “bone”, but unless otherwise specified, the “key point” corresponds to the “joint” of a person, and a “bone” corresponds to the “bone” of the person.

### Overview of Example Embodiments

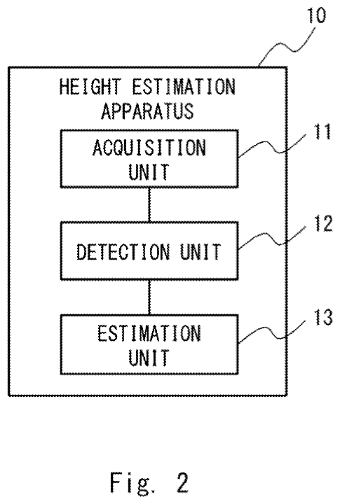

FIG. 2 shows an overview of a height estimation apparatus 10 according to the example embodiment. As shown in FIG. 2, the height estimation apparatus 10 includes an acquisition unit 11, a detection unit 12, and an estimation unit 13.

The acquisition unit 11 acquires a two-dimensional image obtained by capturing an animal such as a person. The detection unit 12 detects a two-dimensional skeletal structure of the animal based on the two-dimensional image acquired by the acquisition unit 11. The estimation unit 13 estimates the height of the animal in a three-dimensional real world based on the two-dimensional skeletal structure detected by the detection unit 12 and an imaging parameter of the two-dimensional image.

Thus, in the example embodiments, a two-dimensional skeletal structure of an animal such as a person is detected from a two-dimensional image, and a height of the animal in a real world is estimated based on the two-dimensional skeletal structure, whereby the height of the animal can be accurately estimated regardless of a posture of the animal.

### First Example Embodiment

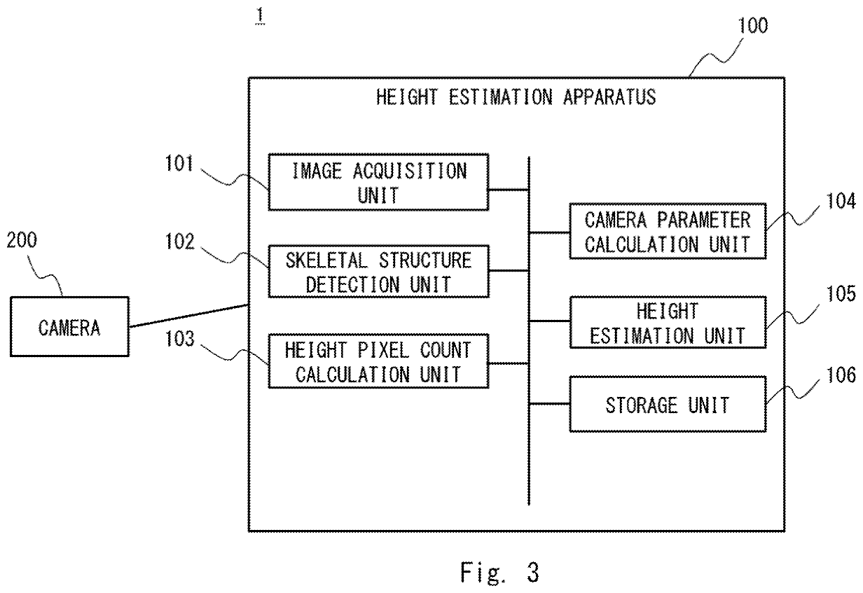

A first example embodiment will be described below with reference to the drawings. FIG. 3 shows a configuration of the height estimation apparatus 100 according to this example embodiment. The height estimation apparatus 100 and a camera 200 constitute a height estimation system 1. For example, the height estimation apparatus 100 and the height estimation system 1 are applied to a monitoring method in a monitoring system as shown in FIG. 1, and a height as an attribute of a person is estimated, a person having the attribute is monitored, and other processing is performed. The camera 200 may be included inside the height estimation apparatus 100.

As shown in FIG. 3, the height estimation apparatus 100 includes an image acquisition unit 101, a skeletal structure detection unit 102, a height pixel count calculation unit 103, a camera parameter calculation unit 104, a height estimation unit 105, and a storage unit 106. A configuration of each unit, i.e., each block, is an example, and may be composed of other units, as long as the method or an operation described later is possible. The height pixel count calculation unit 103 and the height estimation unit 105 may be used as estimation units for estimating a height of a person. Further, the height estimation apparatus 100 is implemented by, for example, a computer apparatus such as a personal computer or a server for executing a program, and instead may be implemented by one apparatus or a plurality of apparatuses on a network.

The storage unit 106 stores information and data necessary for the operation and processing of the height estimation apparatus 100. For example, the storage unit 106 may be a non-volatile memory such as a flash memory or a hard disk apparatus. The storage unit 106 stores images acquired by the image acquisition unit 101, images processed by the skeletal structure detection unit 102, data for machine learning, and so on. The storage unit 106 may be an external storage apparatus or an external storage apparatus on the network. That is, the height estimation apparatus 100 may acquire necessary images, data for machine learning, and so on from the external storage apparatus.

The image acquisition unit 101 acquires a two-dimensional image captured by the camera 200 from the camera 200 which is connected to the height estimation apparatus 100 in a communicable manner. The camera 200 is an imaging unit such as a monitoring camera for capturing a person, and the image acquisition unit 101 acquires, from the camera 200, an image obtained by capturing the person.

The skeletal structure detection unit 102 detects a two-dimensional skeletal structure of the person in the image based on the acquired two-dimensional image. The skeletal structure detection unit 102 detects the skeletal structure of the person based on the characteristics such as joints of the person to be recognized using a skeleton estimation technique by means of machine learning. The skeletal structure detection unit 102 uses, for example, the skeleton estimation technique such as OpenPose of Non Patent Literature 1.

The height pixel count calculation unit 103 calculates the height, which is referred to as a height pixel count, of the person standing upright in the two-dimensional image based on the detected two-dimensional skeletal structure. The height pixel count can be said to be the height of the person in the two-dimensional image, i.e., the length of the whole body of the person in a two-dimensional image space. The height pixel count calculation unit 103 obtains the height pixel count, i.e., a pixel count, from the length, which is the length in the two-dimensional image space, of each bone of the detected skeletal structure. In this example embodiment, the height pixel count is obtained by summing up the lengths of respective bones from the head to the foot of the skeletal structure. When the skeletal structure detection unit 102, by means of the skeleton estimation technique, does not output the top of the head and the foot, the height pixel count may be corrected by multiplying the height pixel count by a constant as necessary.

The camera parameter calculation unit 104 calculates camera parameters, which are imaging conditions of the camera 200, based on the image captured by the camera 200. The camera parameters are imaging parameters of the image and are parameters for converting the length in the two-dimensional image into the length in a three-dimensional real world. For example, the camera parameters include a posture, a position, an imaging angle, a focal length, and the like of the camera 200. An image of an object whose length is known in advance is captured by the camera 200, and then the camera parameters can be obtained from the image.

The height estimation unit 105 estimates the height of the person in the three-dimensional real world based on the calculated camera parameters and the height pixel count in the two-dimensional image. The height estimation unit 105 obtains a relationship between the length of pixel in the image and the length in the real world from the camera parameters, and converts the height pixel count into the height of person in the real world.

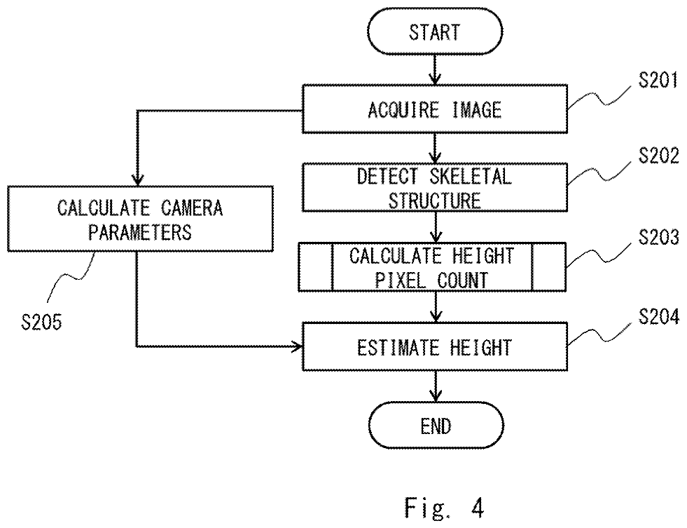

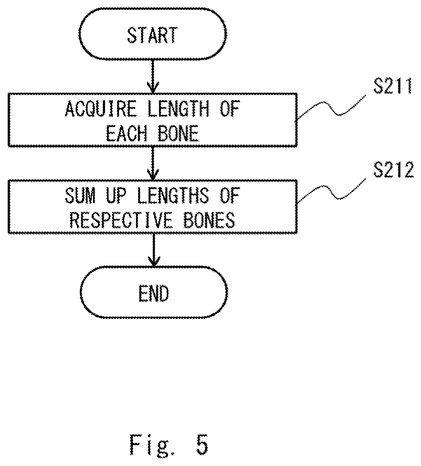

FIGS. 4 and 5 show the operation of the height estimation apparatus 100 according to this example embodiment. FIG. 4 shows a flow from image acquisition to height estimation in the height estimation apparatus 100. FIG. 5 shows a flow of height pixel count calculation processing (S203) in FIG. 4.

As shown in FIG. 4, the height estimation apparatus 100 acquires an image from the camera 200 (S201). The image acquisition unit 101 acquires the image obtained by capturing a person for detecting a skeletal structure, and acquires an image obtained by capturing an object of a predetermined length for calculating the camera parameters.

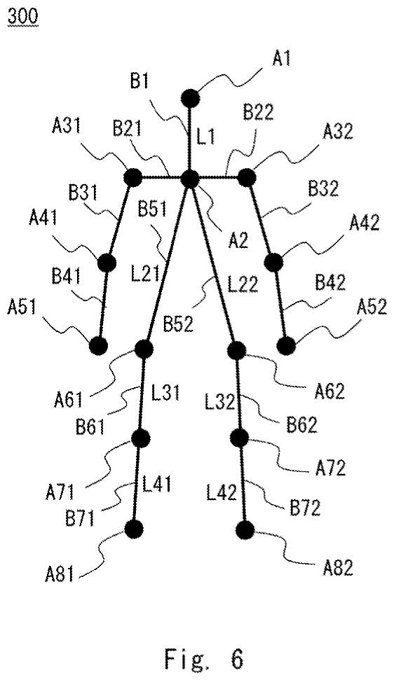

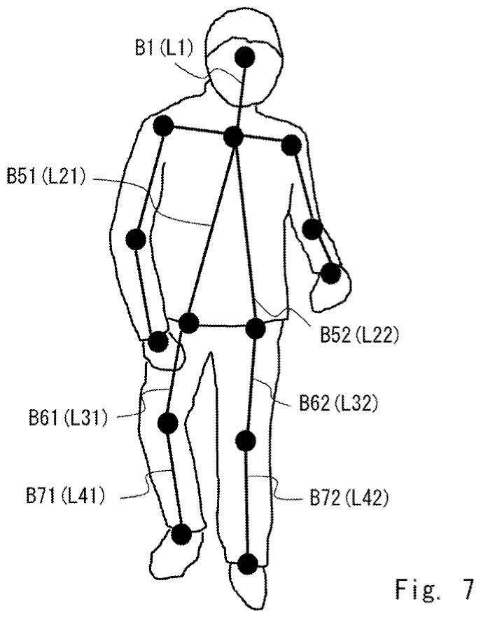

Next, the height estimation apparatus 100 detects the skeletal structure of the person based on the acquired image of the person (S202). FIG. 6 shows the skeletal structure of a human body model 300 detected at this time. FIGS. 7 to 9 show examples of detection of the skeletal structure. The skeletal structure detection unit 102 detects the skeletal structure of the human body model 300, which is a two-dimensional skeleton model, shown in FIG. 6 from the two-dimensional image by the skeleton estimation technique such as OpenPose. The human body model 300 is a two-dimensional model composed of key points such as joints of a person and bones connecting the key points.

The skeletal structure detection unit 102 extracts, for example, characteristic points that can be the key points from the image, and detects each key point of the person by referring to information obtained by machine learning the image of the key point. In the example of FIG. 6, as the key points of a person, a head A1, a neck A2, a right shoulder A31, a left shoulder A32, a right elbow A41, a left elbow A42, a right hand A51, a left hand A52, a right hip A61, a left hip A62, a right knee A71, a left knee A72, a right foot A81, and a left foot A82 are detected. Further, as the bones of the person connecting these key points, a bone B1 connecting the head A1 to the neck A2, bones B21 and B22 respectively connecting the neck A2 to the right shoulder A31 and the neck A2 to the left shoulder A32, bones B31 and B32 respectively connecting the right shoulder A31 to the right elbow A41 and the left shoulder A32 to the left elbow A42, bones B41 and B42 respectively connecting the right elbow A41 to the right hand A51 and the left elbow A42 to the left hand A52, bones B51 and B52 respectively connecting the neck A2 to the right hip A61 and the neck A2 to the left hip A62, bones B61 and B62 respectively connecting the right hip A61 to the right knee A71 and the left hip A62 to the left knee A72, bones B71 and B72 respectively connecting the right knee A71 to the right foot A81 and the left knee A72 to the left foot A82 are detected.

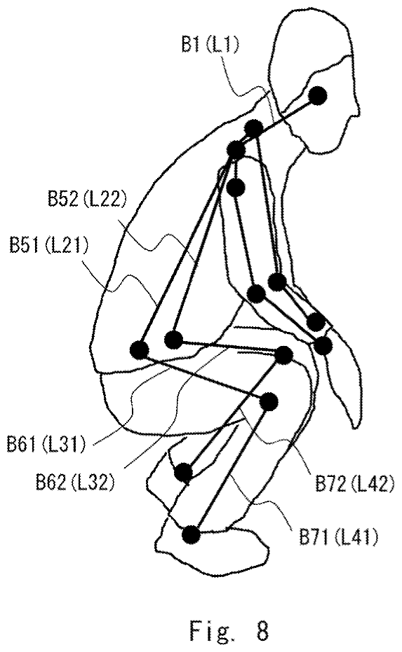

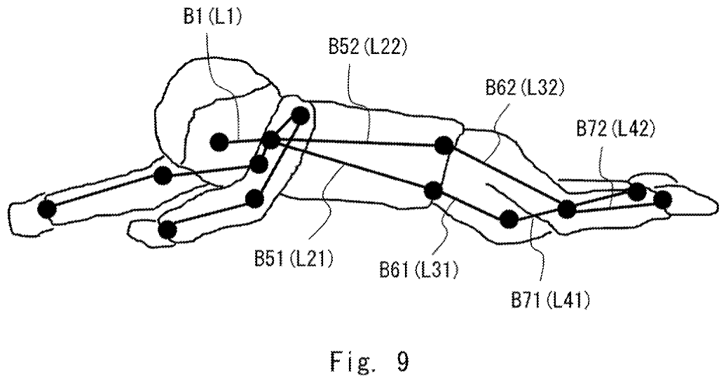

FIG. 7 shows an example in which a person standing upright is detected. In FIG. 7, an image of an upright person is captured from the front, the bones B1, B51, and B52, the bones B61 and B62, and the bones B71 and B72 viewed from the front are detected with no overlapping between them, and the bones B61 and B71 of the right foot are bent slightly more than bones B62 and B72 of the left foot. FIG. 8 shows an example in which a person crouching down is detected. In FIG. 8, an image of the person crouching down is captured from the right side, the bone B1, the bones B51 and B52, the bones B61 and B62, and the bones B71 and B72 viewed from the right side are detected, and the bones B61 and B71 of the right foot and the bones B62 and B72 of the left foot are largely bent and overlapped. FIG. 9 shows an example in which a person lying down is detected. In FIG. 9, an image of the person lying down is captured from diagonally forward left, and the bone B1, the bones B51 and B52, the bones B61 and B62, and the bones B71 and B72 viewed from diagonally forward left are detected, and the bones B61 and B71 of the right foot and the bones B62 and B72 of the left foot are bent and overlapped.

Next, the height estimation apparatus 100 performs the height pixel count calculation processing based on the detected skeletal structure (S203). In the height pixel count calculation processing, as shown in FIG. 5, the height pixel count calculation unit 103 acquires the lengths of the respective bones (S211), and sums up the acquired lengths of the respective bones (S212). The height pixel count calculation unit 103 acquires the lengths of the bones from the head part to the foot part of the person in the two-dimensional image to obtain the height pixel count. That is, from among the bones shown in FIG. 6, the respective lengths, i.e., the pixel count, of the bone B1 (length L1), the bone B51 (length L21), the bone B61 (length L31) and the bone B71 (length L41), or the bone B1 (length L1), the bone B52 (length L22), the bone B62 (length L32), and the bone B72 (length L42) are acquired from the image in which the skeletal structure is detected. The length of each bone can be obtained from the coordinates of each key point in the two-dimensional image. The sum of these values, L1+L21+L31+L41 or L1+L22+L32+L42, multiplied by a correction constant, is calculated as the height pixel count. When both values can be calculated, for example, the larger value is used as the height pixel count. That is, the length of each bone in the image becomes the longest when the image is captured from the front, and is displayed shorter when the bone is tilted in a depth direction with respect to the camera. Therefore, a longer bone is more likely to be captured from the front, and is considered to be closer to an actual value. For this reason, it is preferable that the larger value be selected.

In the example of FIG. 7, the bone B1, the bones B51 and B52, the bones B61 and B62, and the bones B71 and B72 are detected with no overlapping between them. The sums of these bones L1+L21+L31+L41 and L1+L22+L32+L42 are obtained, and for example, a value calculated by multiplying the sum of L1+L22+L32+L42 for the left foot side, which indicates a longer length of the detected bones, by the correction constant is used as the height pixel count.

In the example of FIG. 8, the bone B1, the bones B51 and B52, the bones B61 and B62, and the bones B71 and B72 are detected, and the bones B61 and B71 of the right foot overlap the bones B62 and B72 of the left foot. The sums of these bones L1+L21+L31+L41 and L1+L22+L32+L42 are obtained, and for example, a value calculated by multiplying the sum of L1+L21+L31+L41 for the right foot side, which indicates a longer length of the detected bones, by the correction constant is used as the height pixel count.

In the example of FIG. 9, the bone B1, the bones B51 and B52, the bones B61 and B62, and the bones B71 and B72 are detected, and the bones B61 and B71 of the right foot overlap the bones B62 and B72 of the left foot. The sums of these bones L1+L21+L31+L41 and L1+L22+L32+L42 are obtained, and for example, a value calculated by multiplying the sum of L1+L22+L32+L42 for the left foot side, which indicates a longer length of the detected bones, by the correction constant is used as the height pixel count.

In the meantime, as shown in FIG. 4, the height estimation apparatus 100 calculates the camera parameters based on the image captured by the camera 200 (S205). The camera parameter calculation unit 104 extracts an object whose length is known in advance from a plurality of images captured by the camera 200, and obtains the camera parameters from the size, i.e., pixel count, of the extracted object. The camera parameters may be obtained in advance, and the obtained camera parameters may be acquired if necessary.

Next, the height estimation apparatus 100 estimates the height of the person based on the height pixel count and the camera parameters (S204). The height estimation unit 105 obtains, from the camera parameters, the length in the three-dimensional real world with respect to one pixel in an area where the person is present in the two-dimensional image, namely, the actual length of the pixel unit. In particular, since the length in the real world with respect to one pixel in the image varies depending on the location in the image, the “length in the real world per pixel in the area where the person is present” in the image is obtained. The height pixel count is converted into the height from the obtained actual length of the pixel unit. For example, in FIG. 8, if the sum of the lengths of the bones B1, B51, B61, and B71 is L1+L21+L31+L41=100 pixels, and 1 pixel=1.7 cm in the area where the person is present, the height is 170 cm.

As described above, in this example embodiment, the skeletal structure of the person is detected from the two-dimensional image, the height pixel count is obtained by summing up the lengths of the bones in the two-dimensional image of the detected skeletal structure. Further, the height of the person in the real world is estimated in consideration of the camera parameters. The height can be obtained by summing the lengths of the bones from head to foot, and thus the height can be estimated in a simple way. In addition, since it is sufficient to detect at least the skeleton from the head to the foot by the skeleton estimation technique by means of machine learning, the height can be estimated with high accuracy even when the whole body of the person does not necessarily appear in the image such as when the person is crouching down.

### Second Example Embodiment

Next, a second example embodiment will be described. In this example embodiment, in the height pixel count calculation processing according to the first example embodiment, the height pixel count is calculated using a human body model showing a relationship between a length of each bone and a length of a whole body, i.e., a height in the two-dimensional image space. The processing other than the height pixel count calculation processing is the same as that of the first example embodiment.

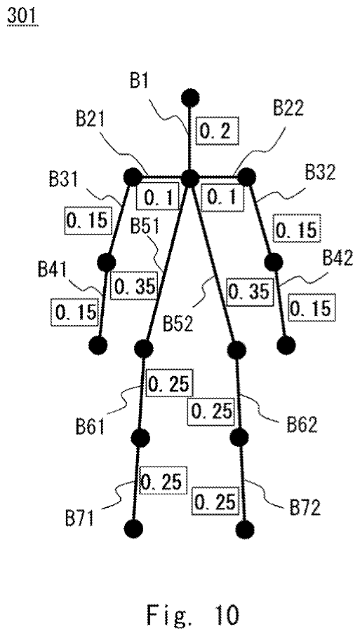

FIG. 10 shows a human body model 301, i.e., a two-dimensional skeleton model, showing the relationship between the length of each bone in the two-dimensional image space and the length of the whole body in the two-dimensional image space used in this example embodiment. As shown in FIG. 10, the relationship between the length of each bone of an average person and the length of the whole body, which is a ratio of the length of each bone to the length of the whole body, is associated with each bone of the human body model 301. For example, the length of the bone B1 of the head is the total length×0.2 (20%), the length of the bone B41 of the right hand is the total length×0.15 (15%), and the length of the bone B71 of the right foot is the total length×0.25 (25%). By storing such information of the human body model 301 in the storage unit 106, the average length of the whole body, i.e., the pixel count, can be obtained from the length of each bone. In addition to a human body model of an average person, a human body model may be prepared for each attribute of the person such as age, gender, nationality, etc. By doing so, the length, namely, the height, of the whole body can be appropriately obtained according to the attribute of the person.

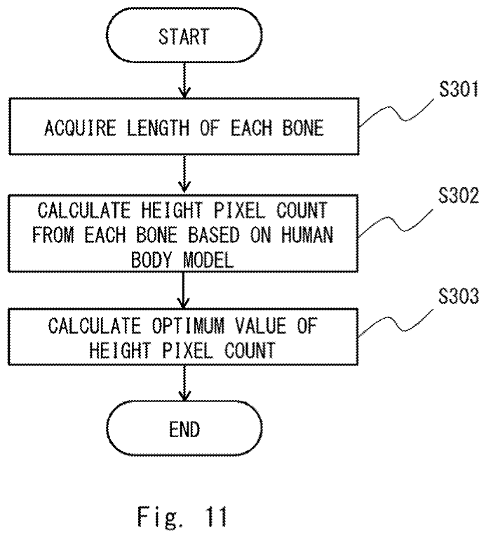

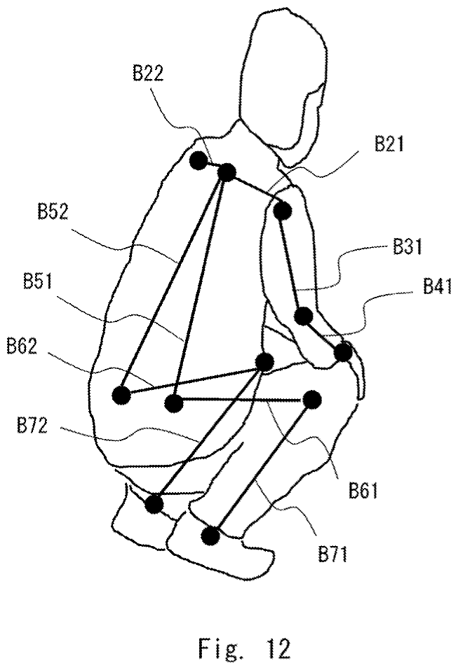

FIG. 11 shows processing for calculating the height pixel count according to this example embodiment, and shows a flow of the height pixel count calculation processing (S203) shown in FIG. 4 according to the first example embodiment. In the height pixel count calculation processing according to this example embodiment, as shown in FIG. 11, the height pixel count calculation unit 103 acquires the length of each bone (S301). In the skeletal structure detected as in the first example embodiment, the height pixel count calculation unit 103 acquires the lengths of all bones, which are the lengths of the bones in the two-dimensional image space. FIG. 12 shows an example in which the skeletal structure is detected by capturing an image of a person crouching down from diagonally backward right. In this example, the bone of the head and the bones of the left arm and the left hand cannot be detected, because the face and the left side of the person do not appear in the image. Therefore, the lengths of the detected bones B21, B22, B31, B41, B51, B52, B61, B62, B71, and B72 are acquired.

Next, the height pixel count calculation unit 103 calculates the height pixel count from the length of each bone based on the human body model (S302). The height pixel count calculation unit 103 obtains the height pixel count from the length of each bone with reference to the human body model 301 showing the relationship between each bone and the length of the whole body as shown in FIG. 10. For example, since the length of the bone B41 of the right hand is the length of the whole body×0.15, the height pixel count based on the bone B41 is obtained by calculating the length of the bone B41/0.15. Further, since the length of the bone B71 of the right foot is the length of the whole body×0.25, the height pixel count based on the bone B71 is obtained by calculating the length of the bone B71/0.25.

The human body model to be referred to here is, for example, a human body model of an average person, but the human body model may be selected according to the attributes of the person such as age, gender, nationality, etc. For example, when a face of a person appears in the captured image, an attribute of the person is identified based on the face, and a human body model corresponding to the identified attribute is referred to. By referring to the information obtained by machine learning the face for each attribute, the attribute of the person can be recognized from the characteristics of the face of the image. When the attribute of the person cannot be identified from the image, a human body model of an average person may be used.

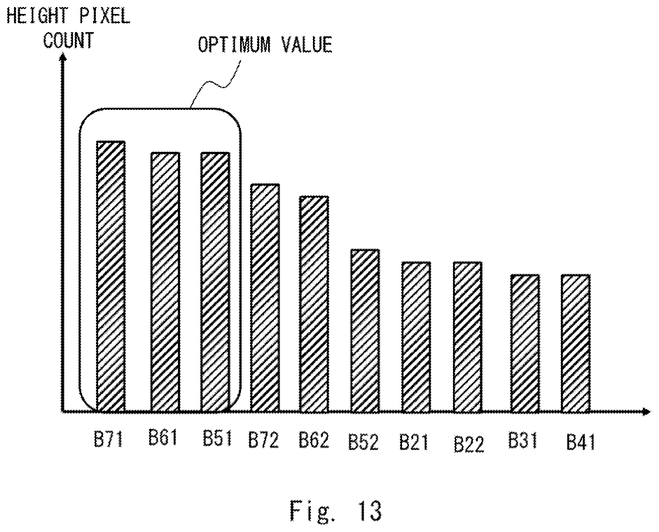

Next, the height pixel count calculation unit 103 calculates an optimum value of the height pixel count (S303). The height pixel count calculation unit 103 calculates the optimum value of the height pixel count from the height pixel count obtained for each bone. For example, as shown in FIG. 13, a histogram of the height pixel count obtained for each bone is generated, and a large height pixel count is selected from the histogram. That is, among the plurality of height pixel counts obtained based on the plurality of bones, the height pixel count larger than the others is selected. For example, the top 30% height pixel counts are defined as valid values. In such a case, in FIG. 13, the height pixel counts calculated based on the bones B71, B61, and B51 are selected. The average of the selected height pixel counts may be obtained as the optimum value, or the maximum height pixel count may be used as the optimum value. Since the height is obtained from the length of the bone in the two-dimensional image, when the image of the bone is not captured from the front, that is, when the image of the bone is captured tilted in the depth direction with respect to the camera, the length of the bone becomes shorter than the length of the bone captured from the front. For this reason, a larger height pixel count is more likely to be calculated from the length of the bone captured from the front compared to a smaller height pixel count, and thus the larger height pixel count indicates a more likely value (greater likelihood). Thus, the larger height pixel count is used as the optimum value.

As described above, in this example embodiment, the height of the person in the real world is estimated by obtaining the height pixel count based on the bones of the detected skeletal structure using the human body model showing the relationship between the bones in the two-dimensional image space and the length of the whole body. In this way, even when all the skeletons from the head to the foot cannot be acquired, the height can be estimated from some of the bones. In particular, by employing a larger value of the height, i.e., a larger height pixel count, which is obtained from a plurality of bones, the height can be accurately estimated.

### Third Example Embodiment

Next, a third example embodiment will be described. In this example embodiment, instead of the height pixel count calculation processing and the height estimation processing according to the first example embodiment, a height in the real world is estimated by fitting a three-dimensional human body model to a two-dimensional skeletal structure. Other aspects are the same as those of the first example embodiment.

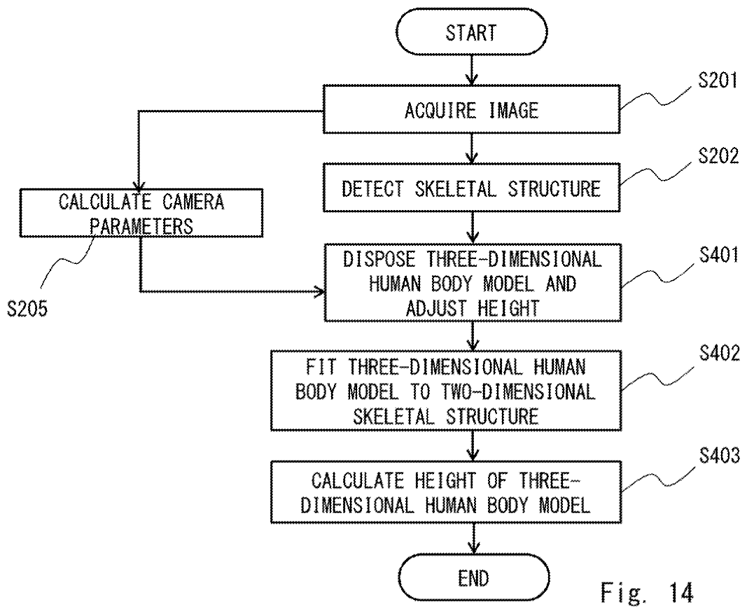

FIG. 14 shows a flow of the height estimation processing according to this example embodiment. In the height estimation processing according to this example embodiment, as shown in FIG. 14, the height estimation apparatus 100 first acquires a two-dimensional image from the camera 200 (S201), detects a two-dimensional skeletal structure of a person in the image (S202), and calculates camera parameters (S205), in a manner similar to FIG. 4 of the first example embodiment. Next, the height estimation unit 105 of the height estimation apparatus 100 disposes a three-dimensional human body model and adjusts a height of the a three-dimensional human body model (S401). The height estimation unit 105 prepares the three-dimensional human body model for calculating the height for the two-dimensional skeletal structure detected as in the first example embodiment, and disposes the three-dimensional human body model in the same two-dimensional image as the two-dimensional image used for detecting the two-dimensional skeletal structure based on the camera parameters. Specifically, “a relative positional relationship between the camera and the person in the real world” is specified from the camera parameters and the two-dimensional skeletal structure. For example, assuming that the position of the camera is at coordinates (0, 0, 0), the coordinates (x, y, z) of the position where the person stands or sits are specified. An image obtained by disposing the three-dimensional human body model at the same position (x, y, z) as that of the specified person is assumed and the image is captured, so that the two-dimensional skeletal structure and the three-dimensional human body model are superimposed.

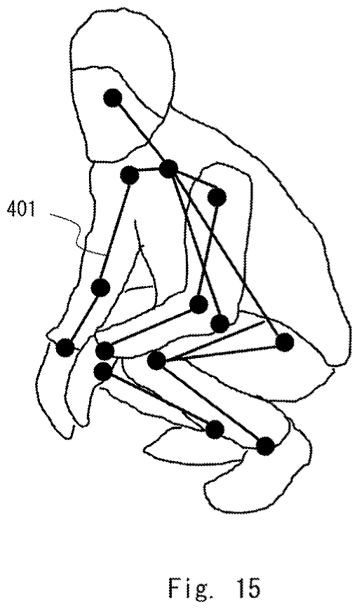

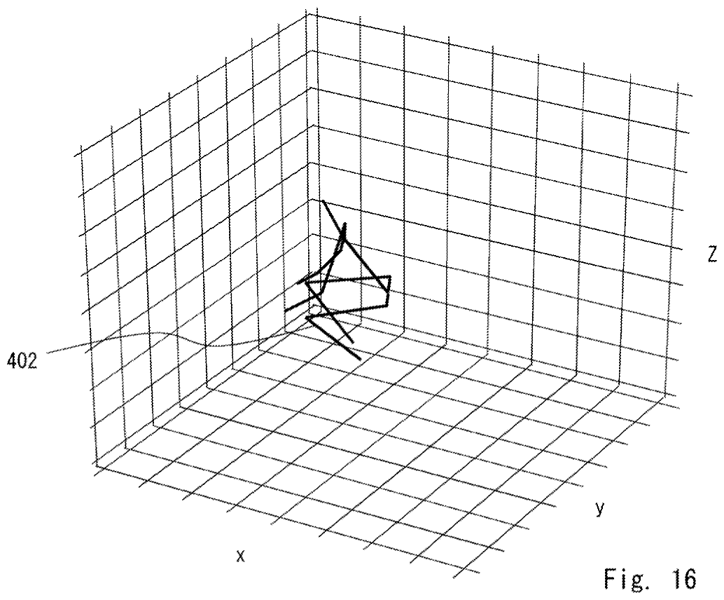

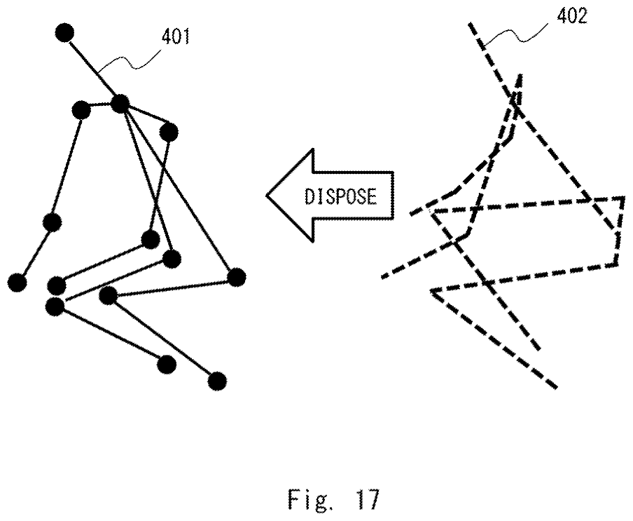

FIG. 15 shows an example in which a person crouching down is captured from diagonally forward left to detect the two-dimensional skeletal structure 401. The two-dimensional skeletal structure 401 has two-dimensional coordinate information. It is preferable that all bones be detected, but some bones may not be detected. A three-dimensional human body model 402 as shown in FIG. 16 is prepared for the two-dimensional skeletal structure 401. The three-dimensional human body model, i.e., three-dimensional skeleton model, 402 has three-dimensional coordinate information and is a skeleton model having the same shape as that of the two-dimensional skeletal structure 401. Next, as shown in FIG. 17, the prepared three-dimensional human body model 402 is disposed and superimposed on the detected two-dimensional skeletal structure 401. The three-dimensional human body model 402 is superimposed and also adjusted so that the height of the three-dimensional human body model 402 fits to the two-dimensional skeletal structure 401.

The three-dimensional human body model 402 prepared here may be a model in a state close to the posture of the two-dimensional skeletal structure 401 as shown in FIG. 17 or a model in an upright state. For example, a technique for estimating the posture of the three-dimensional space from the two-dimensional image using the machine learning may be used to generate the three-dimensional human body model 402 of the estimated posture. By learning the information about the joints of the two-dimensional image and the joints of the three-dimensional space, the three-dimensional posture can be estimated from the two-dimensional image.

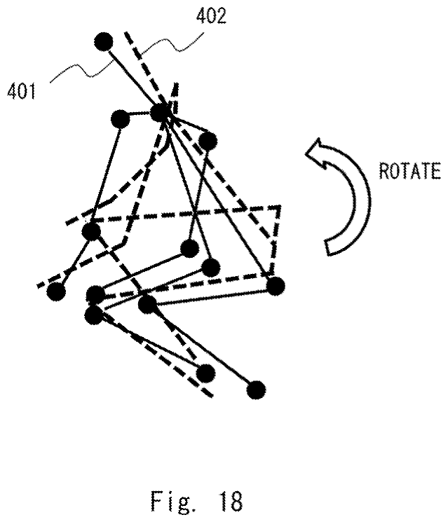

Next, the height estimation unit 105 fits the three-dimensional human body model to the two-dimensional skeletal structure (S402). As shown in FIG. 18, the height estimation unit 105 deforms the three-dimensional human body model 402 so that the three-dimensional human body model 402 and the two-dimensional skeletal structure 401 have the same posture when the three-dimensional human body model 402 is superimposed on the two-dimensional skeletal structure 401. That is, the height, the orientation of the body, and the angles of the joints of the three-dimensional human body model 402 are adjusted and optimized so that there is no difference between the three-dimensional human body model 402 and the two-dimensional skeletal structure 401. For example, the joints of the three-dimensional human body model 402 are rotated within a movable range of the person, and the entire three-dimensional human body model 402 is rotated or the entire size thereof is adjusted. The fitting of the three-dimensional human body model and the two-dimensional skeletal structure is performed in a two-dimensional space, i.e., on the two-dimensional coordinates. That is, the three-dimensional human body model is mapped to the two-dimensional space, and the three-dimensional human body model is optimized to the two-dimensional skeletal structure in consideration of how the deformed three-dimensional human body model changes in the two-dimensional space, i.e., on the two-dimensional image.

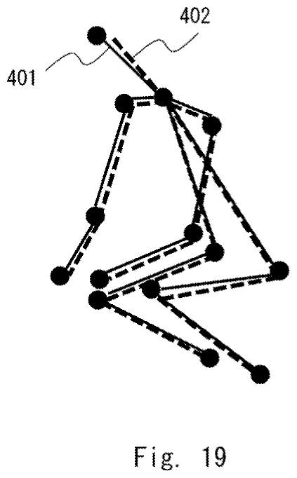

Next, the height estimation unit 105 calculates the height of the fitted three-dimensional human body model (S403). As shown in FIG. 19, when the difference between the three-dimensional human body model 402 and the two-dimensional skeletal structure 401 is eliminated and the posture of the three-dimensional human body model 402 matches the posture of the two-dimensional skeletal structure 401, the height estimation unit 105 obtains the height of the three-dimensional human body model 402 in this state. Note that since the height of the three-dimensional human body model when the optimization is completed is used as it is as the height in the real world to be obtained, for example, the height in the unit of centimeters, it is not necessary to calculate the height pixel count in this example embodiment unlike the first and second example embodiments. For example, the height is calculated from the lengths of the bones from the head to the foot when the three-dimensional human body model 402 is made to stand upright. In the manner similar to the first example embodiment, the lengths of the bones from the head to the foot of the three-dimensional human body model 402 may be summed.

As described above, in this example embodiment, the three-dimensional human body model is fitted to the two-dimensional skeletal structure based on the camera parameters, and the height of the person in the real world is estimated based on the three-dimensional human body model. Specifically, the height of the fitted three-dimensional human body model is used as it is as the estimated height. In this manner, even when all bones do not face the front in the image, that is, even when all bones are viewed diagonally and there is a large difference from actual lengths of the bones, the height can be accurately estimated. When the method according to the first to the third example embodiments is applicable, all of the methods or a combination of the methods may be used to obtain the height. In this case, a value closer to the average height of the person may be used as the optimum value.

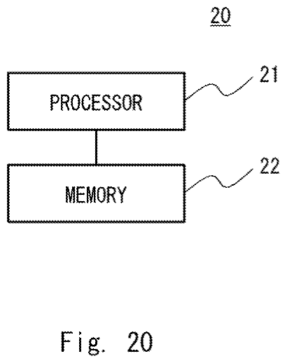

Note that each of the configurations in the above-described example embodiments is constituted by hardware and/or software, and may be constituted by one piece of hardware or software, or may be constituted by a plurality of pieces of hardware or software. The functions and processing of the height estimation apparatuses 10 and 100 may be implemented by a computer 20 including a processor 21 such as a Central Processing Unit (CPU) and a memory 22 which is a storage device, as shown in FIG. 20. For example, a program, i.e., a height estimation program, for performing the method according to the example embodiments may be stored in the memory 22, and each function may be implemented by the processor 21 executing the program stored in the memory 22.

These programs can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Further, the present disclosure is not limited to the above-described example embodiments and may be modified as appropriate without departing from the purpose thereof. For example, although a height of a person is estimated in the above description, a height of an animal other than a person having a skeletal structure such as mammals, reptiles, birds, amphibians, fish, etc. may be estimated.

Although the present disclosure has been described above with reference to the example embodiments, the present disclosure is not limited to the example embodiments described above. The configurations and details of the present disclosure may be modified in various ways that would be understood by those skilled in the art within the scope of the present disclosure.

The whole or part of the example embodiments disclosed above can be described as, but not limited to, the following supplementary notes.

### (Supplementary Note 1)

A height estimation apparatus comprising:

acquisition means for acquiring a two-dimensional image obtained by capturing an animal;

detection means for detecting a two-dimensional skeletal structure of the animal based on the acquired two-dimensional image; and

estimation means for estimating a height of the animal in a three-dimensional real world based on the detected two-dimensional skeletal structure and an imaging parameter of the two-dimensional image.

### (Supplementary Note 2)

The height estimation apparatus according to Supplementary note 1, wherein

the estimation means estimates the height based on a length of a bone in a two-dimensional image space included in the two-dimensional skeletal structure.

### (Supplementary Note 3)

The height estimation apparatus according to Supplementary note 2, wherein

the estimation means estimates the height based on a sum of the lengths of the bones from a foot to a head included in the two-dimensional skeletal structure.

### (Supplementary Note 4)

The height estimation apparatus according to Supplementary note 2, wherein

the estimation means estimates the height based on a two-dimensional skeleton model showing a relationship between the length of the bone and a length of a whole body of the animal in the two-dimensional image space.

### (Supplementary Note 5)

The height estimation apparatus according to Supplementary note 4, wherein

the estimation means estimates the height based on the two-dimensional skeleton model corresponding to an attribute of the animal.

### (Supplementary Note 6)

The height estimation apparatus according to Supplementary note 4 or 5, wherein

the estimation means estimates the height based on a tallest height from among a plurality of the heights obtained based on the plurality of bones in the two-dimensional skeletal structure.

### (Supplementary Note 7)

The height estimation apparatus according to Supplementary note 1, wherein

the estimation means estimates the height based on a three-dimensional skeleton model fitted to the two-dimensional skeletal structure based on the imaging parameter.

### (Supplementary Note 8)

The height estimation apparatus according to Supplementary note 7, wherein

the estimation means uses a height of the fitted three-dimensional skeleton model as the estimated height.

### (Supplementary Note 9)

A height estimation method comprising:

acquiring a two-dimensional image obtained by capturing an animal;

detecting a two-dimensional skeletal structure of the animal based on the acquired two-dimensional image; and

estimating a height of the animal in a three-dimensional real world based on the detected two-dimensional skeletal structure and an imaging parameter of the two-dimensional image.

### (Supplementary Note 10)

The height estimation method according to Supplementary note 9, wherein

in the estimation of the height, the height is estimated based on a length of a bone in a two-dimensional image space included in the two-dimensional skeletal structure.

### (Supplementary Note 11)

A height estimation program for causing a computer to execute processing of:

acquiring a two-dimensional image obtained by capturing an animal;

detecting a two-dimensional skeletal structure of the animal based on the acquired two-dimensional image; and

estimating a height of the animal in a three-dimensional real world based on the detected two-dimensional skeletal structure and an imaging parameter of the two-dimensional image.

### (Supplementary Note 12)

The height estimation program according to Supplementary note 11, wherein

in the estimation of the height, the height is estimated based on a length of a bone in a two-dimensional image space included in the two-dimensional skeletal structure.

### (Supplementary Note 13)

A height estimation system comprising:

a camera; and

a height estimation apparatus, wherein the height estimation apparatus comprises:

acquisition means for acquiring, from the camera, a two-dimensional image obtained by capturing an animal;

detection means for detecting a two-dimensional skeletal structure of the animal based on the acquired two-dimensional image; and

estimation means for estimating a height of the animal in a three-dimensional real world based on the detected two-dimensional skeletal structure and an imaging parameter of the two-dimensional image.

### (Supplementary Note 14)

The height estimation apparatus according to Supplementary note 13, wherein

the estimation means estimates the height based on a length of a bone in a two-dimensional image space included in the two-dimensional skeletal structure.

### REFERENCE SIGNS LIST

1 HEIGHT ESTIMATION SYSTEM
10 HEIGHT ESTIMATION APPARATUS
11 ACQUISITION UNIT
12 DETECTION UNIT
13 ESTIMATION UNIT
20 COMPUTER
21 PROCESSOR
22 MEMORY
100 HEIGHT ESTIMATION APPARATUS
101 IMAGE ACQUISITION UNIT
102 SKELETAL STRUCTURE DETECTION UNIT
103 HEIGHT PIXEL COUNT CALCULATION UNIT
104 CAMERA PARAMETER CALCULATION UNIT
105 HEIGHT ESTIMATION UNIT
106 STORAGE UNIT
200 CAMERA
300, 301 HUMAN BODY MODEL
401 TWO-DIMENSIONAL SKELETAL STRUCTURE
402 THREE-DIMENSIONAL HUMAN BODY MODEL

What is claimed is:

1. A height estimation apparatus comprising:
at least one memory storing instructions, and
at least one processor configured to execute the instructions stored in the at least one memory to;
acquire a two-dimensional image obtained by capturing an animal;
detect a two-dimensional skeletal structure of the animal based on the acquired two-dimensional image; and
estimate a height of the animal in a three-dimensional real world based on the detected two-dimensional skeletal structure and an imaging parameter of the two-dimensional image.

2. The height estimation apparatus according to claim 1, wherein
the at least one processor is further configured to execute the instructions stored in the at least one memory to estimate the height based on a length of a bone in a two-dimensional image space included in the two-dimensional skeletal structure.

3. The height estimation apparatus according to claim 2, wherein
the at least one processor is further configured to execute the instructions stored in the at least one memory to estimate the height based on a sum of the lengths of the bones from a foot to a head included in the two-dimensional skeletal structure.

4. The height estimation apparatus according to claim 2, wherein
the at least one processor is further configured to execute the instructions stored in the at least one memory to estimate the height based on a two-dimensional skeleton model showing a relationship between the length of the bone and a length of a whole body of the animal in the two-dimensional image space.

5. The height estimation apparatus according to claim 4, wherein
the at least one processor is further configured to execute the instructions stored in the at least one memory to estimate the height based on the two-dimensional skeleton model corresponding to an attribute of the animal.

6. The height estimation apparatus according to claim 1, wherein
the at least one processor is further configured to execute the instructions stored in the at least one memory to estimate the height based on a tallest height from among a plurality of the heights obtained based on the plurality of bones in the two-dimensional skeletal structure.

7. The height estimation apparatus according to claim 1, wherein
the at least one processor is further configured to execute the instructions stored in the at least one memory to estimate the height based on a three-dimensional skeleton model fitted to the two-dimensional skeletal structure based on the imaging parameter.

8. The height estimation apparatus according to claim 7, wherein
the at least one processor is further configured to execute the instructions stored in the at least one memory to use a height of the fitted three-dimensional skeleton model as the estimated height.

9. A height estimation method comprising:
acquiring a two-dimensional image obtained by capturing an animal;
detecting a two-dimensional skeletal structure of the animal based on the acquired two-dimensional image; and
estimating a height of the animal in a three-dimensional real world based on the detected two-dimensional skeletal structure and an imaging parameter of the two-dimensional image.

10. A non-transitory computer readable medium storing a program for causing a computer to execute processing of:
acquiring a two-dimensional image obtained by capturing an animal;
detecting a two-dimensional skeletal structure of the animal based on the acquired two-dimensional image; and
estimating a height of the animal in a three-dimensional real world based on the detected two-dimensional skeletal structure and an imaging parameter of the two-dimensional image.
